(19) United States
(12) Patent Application Publication
(10) Pub. No.: US 2011/0245758 A1
(43) Pub. Date: Oct. 6, 2011

(54) TOPICAL ANESTHETIC AND ANTISEPTIC DISPENSING DEVICE

(76) Inventor: Michael Wycoki, Hobe Sound, FL (US)

(21) Appl. No.: 13/164,213

(22) Filed: Jun. 20, 2011

Related U.S. Application Data
(62) Division of application No. 12/388,347, filed on Feb. 18, 2009.

(51) Int. Cl. A61M 37/00

(52) U.S. Cl. 604/22

(57) ABSTRACT

Embodiments of the present invention provide a topical anesthetic and antiseptic dispensing device article of manufacture. In an embodiment of the invention, the article of manufacture includes a penetrating instrument including an elongated body with first and second distal end such that at least one of the distal ends includes a penetrating edge. By way of example, the penetrating instrument can be a syringe, an IV catheter needle assembly, a pressurized tube holder configured with a needle for blood draw, or a scalpel, to name a few possibilities. The article of manufacture also includes a container coupled to the elongated body of the penetrating instrument. The container provides at least one chamber. Finally, the article of manufacture includes a nozzle connected to the container and configured to dispense pressurized content of the container responsive to activating a trigger communicating with the nozzle.

### CROSS-REFERENCE TO RELATED APPLICATION

This application is a divisional patent application of U.S. patent application Ser. No. 12/388,347, filed Feb. 18, 2009, the entirety of which is incorporated herein.

### BACKGROUND OF THE INVENTION

1. Field of the Invention

The present invention relates to the field of anesthetic and anesthetic dispensing devices and more particularly to aerosol and pressurized type topical anesthetic and anesthetic single dose dispensing devices.

2. Description of the Related Art

Penetrating instruments like scalpels, knives, intravenous (IV) needle assemblies and syringes form an integral part of medical care. One cannot imagine health care without the use of a penetrating instrument such as a needle for use in an injection, or a scalpel for use in a surgical procedure. Indeed, even the most simplistic of diagnostic techniques—the blood draw—requires the use of a needle (ordinarily included as part of a pressurized tube holder configured for blood draw). Even still, excessive pain and adverse reactions associated with the use of a penetrating instrument can cause patient anxiety and unnecessary distress. Consequently, health care providers constantly seek methods and tools to alleviate the pain and reaction caused by the use of a penetrating instrument.

In this regard, it is well known that spraying ethyl chloride on a target zone of a patient for a penetrating instrument can greatly reduce penetrating instrument related pain. Specifically, ethyl chloride is a topical anesthetic. When topically applied to the skin, ethyl chloride creates an instantaneous anesthetic effect on the surface of the site caused by the immediate evaporation of the ethyl chloride. As a result, the cooling effect created by spraying ethyl chloride on a target zone of a patient interrupts the ability of the patient to experience pain resulting from the use of the penetrating instrument on the target zone.

Yet, the anesthetic effect of ethyl chloride generally lasts only upwards of a few seconds. Thus, unless the health care provider is quick to apply the penetrating instrument immediately following the application of ethyl chloride, the ethyl chloride may have to be reapplied. Thus, typically a nurse will clean the target area with an antiseptic, position all equipment and instruments ready for the planned procedure and then apply the ethyl chloride immediately before performing the planned procedure. Accordingly, the described protocol of sterilizing the target zone, preparing the equipment and instruments and applying ethyl chloride can increase the overall time a nurse must spend with each patient. Further, generally an additional person is required in order to complete the anesthetic and antiseptic protocol described herein.

Of note, by having a second nurse present to apply the ethyl chloride in concert with a first nurse performing the expected procedure with the use of a penetrating instrument, the overall time spent on a patient can decrease, thus increasing the capacity to perform more procedures on more patients. However, an additional nurse can be costly. Further, coordinating the placement of the ethyl chloride with the anticipated placement of a needle between two nurses can be problematic in that one nurse may not completely recognize the intention of the other nurse in respect to the placement of the penetrating instrument. Even still, having only a single nurse sterilize the target zone, apply the ethyl chloride to the target zone and then perform the planned procedure can result in the unnecessary waste of time when multiple applications of ethyl chloride is required.

### BRIEF SUMMARY OF THE INVENTION

Embodiments of the present invention provide a topical anesthetic and antiseptic dispensing device article of manufacture. In an embodiment of the invention, the article of manufacture includes a penetrating instrument including an elongated body with first and second distal end such that at least one of the distal ends includes a penetrating edge. By way of example, the penetrating instrument can be a syringe, an IV catheter needle assembly, a pressurized tube holder configured with a needle for blood draw, or a scalpel, to name a few possibilities. The article of manufacture also includes a container coupled to the elongated body of the penetrating instrument. The container provides at least one chamber. Finally, the article of manufacture includes a nozzle connected to the container and configured to dispense pressurized content of the container responsive to activating a trigger communicating with the nozzle.

In one aspect of the embodiment of the invention, the chamber contains a pressurized combination of ethyl chloride and alcohol. In another aspect of the embodiment, the chamber contains a pressurized combination of ethyl chloride and povidone-iodine (e.g. BETADINE). In yet another aspect of the embodiment, the container includes two chambers, each chamber containing different pressurized content. In respect to the latter aspect of the embodiment, one chamber contains pressurized ethyl chloride and another chamber contains alcohol. Alternatively, one chamber contains pressurized ethyl chloride and another chamber contains povidone-iodine. Further in respect to the latter aspect of the embodiment, the nozzle can be a bi-valve nozzle with two openings, each of the two openings corresponding to a different one of the two chambers.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1A is a perspective view of a topical anesthetic and antiseptic dispensing device article of manufacture coupled to a syringe.

FIG. 1B is a perspective view of a topical anesthetic and antiseptic dispensing device article of manufacture coupled to a scalpel.

FIG. 1C is a perspective view of a topical anesthetic and antiseptic dispensing device article of manufacture coupled to a pressurized tube holder configured for blood draw.

FIG. 1D is a perspective view of a topical anesthetic and antiseptic dispensing device article of manufacture coupled to an IV catheter.

FIG. 1E is a perspective view of a topical anesthetic and antiseptic dispensing device article of manufacture as a stand-alone hand-held unit.

FIG. 2 is a component part perspective view of a topical anesthetic and antiseptic dispensing device article of manufacture.

FIG. 3 is a side view sequence illustration of a topical anesthetic and antiseptic dispensing device article of manufacture in use.

FIG. 4A is a top view of a dual chamber of a topical anesthetic and antiseptic dispensing device.

FIG. 4B is a cross-sectional view of the interior construction of a dual chamber of a topical anesthetic and antiseptic dispensing device of FIG. 4A.

FIG. 5A is a top view of a single chamber of a topical anesthetic and antiseptic dispensing device.

FIG. 5B is a cross-sectional view of the interior construction of the single chamber of a topical anesthetic and antiseptic dispensing device of FIG. 5A.

### DETAILED DESCRIPTION OF THE INVENTION

Embodiments of the present invention provide a topical anesthetic and antiseptic dispensing device article of manufacture. The dispensing device article of manufacture includes a container with one or more chambers. At least one of the chambers can contain a pressurized chemical providing at least an anesthetic effect when released onto a target zone of a patient, and optionally an antiseptic effect when released onto the target zone. For example, the chemicals can include ethyl chloride or a combination of ethyl chloride and alcohol, or ethyl chloride and povidone-iodine. Optionally, the container can be a modular container accepting one or more canisters of pressurized chemicals such that each canister accounts for a chamber of pressurized chemicals and at least one of the canisters can include a pressurized form of ethyl chloride.

Of note, the container can be adapted for coupling to a penetrating instrument, such as a syringe, scalpel, knife, pressurized tube holder configured for blood draw or an IV catheter needle assembly. In this way, an anesthetic (and optionally an antiseptic) can be released onto the target zone of a patient within close temporal proximity of the use of the coupled penetrating instrument without requiring multiple health care providers to both apply the anesthetic and to use the penetrating instrument.

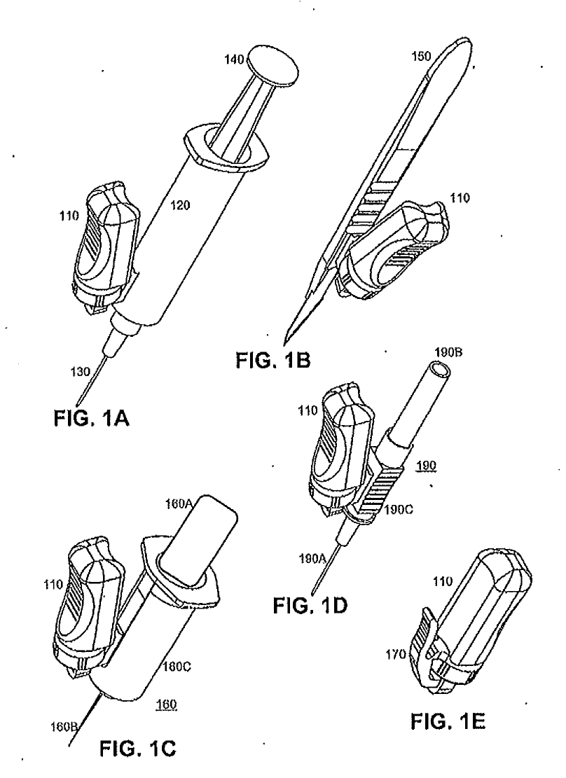

In further illustration, FIGS. 1A through 1D are perspective views of a topical anesthetic and antiseptic dispensing device article of manufacture. As shown in each of FIGS. 1A through 1D, a container 110 can be attached to a penetrating instrument 120, 150, 160, 170 having an elongated body and distal ends opposite to one another with respect to the elongated body. For instance, as shown in FIG. 1A, the penetrating instrument can be a syringe 120 having a pump 140 at one end and a needle 130 at the opposite end. Alternatively, the container 110 can be configured to adapt to a scalpel 150 as shown in FIG. 1B. Another configuration shown in FIG. 1C depicts the container 110 coupled to a pressurized tube holder configured for blood draw 160. The pressurized tube holder configured for blood draw 160 can include a housing 160C with a needle 160B disposed at one end and a pressurized tube 160A at an opposite end partially disposed in the housing 160C for attachment to an opening in the needle so as to facilitate the collection of blood. Yet, another configuration as shown in FIG. 1D can include the container 110 adapted for coupling to an IV catheter needle assembly 190 including a body 190C, a catheter port 190B at one end of the body 190C and a needle 190A at an opposite end of the body 190C.

In all four exemplary aspects of the embodiment illustrated in FIGS. 1A through 1D, the container 110 can include a rotatable joint disposed between the modular canister 110 and the penetrating instrument 120, 150, 160, 170 such that the depression of the container 110 towards the penetrating instrument acts to create an opening between the atmosphere and the pressurized content contained by the container 110 allowing for the pressurized content to be released into the atmosphere and directed onto a target zone of a patient as directed by a nozzle (not shown). Notwithstanding, as shown in FIG. 1E, the container 110 can be a stand-alone hand-held unit having a trigger handle 170 in order to allow the contents of the modular canister 110 to be released out of the modular canister 110 onto a target zone when the trigger handle 170 is rotated toward the container 110.

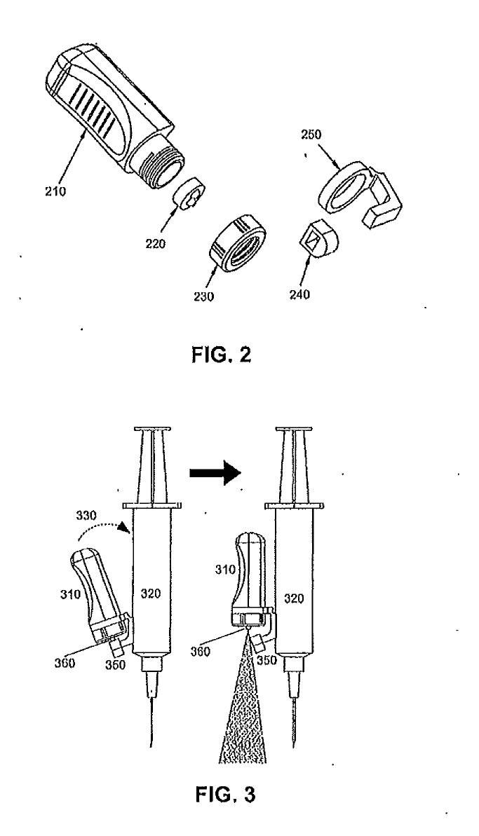

In more particular illustration of an embodiment of the dispensing device article of manufacture, FIG. 2 provides a perspective view of a anesthetic and antiseptic dispensing device article of manufacture. As shown in FIG. 2, the dispensing device article of manufacture can include a container 210 providing at least one chamber containing a pressurized anesthetic. In particular, two separate chambers can be provided, each containing a different chemical, at least one of the chambers containing pressurized ethyl chloride. To facilitate the dispensing of the content of both chambers, a bi-valve nozzle 220 can be attached to the chamber 210 to allow dispensing of the two different chemicals simultaneously. Furthermore, a locking ring 230 with a rubber stop 240 can secure over the bi-valve nozzle 220.

Of note, a trigger 250 can be coupled to the chamber 210 to allow for a pressurized aerosol type triggering mechanism that can evenly dispense the contents of the modular canister 110. Specifically, the trigger 250 can be biased to hold the rubber stop 240 against the bi-valve nozzle 220. When activated, for example rotated, the trigger 250 can permit the rubber stop 240 to release the rubber stop 240 from biased contact against the bi-valve nozzle 220. In consequence, the content of each chamber of the container 210 can be released through the bi-valve nozzle 220 onto a target zone of a patient.

Importantly, the container 210 can be configured for coupling to a penetrating instrument, for example by way of glue or weld or fastener. In illustration, as shown in FIG. 3, the container 310 can be coupled to a penetrating instrument 320 such as a syringe by way of the trigger 350. In this regard, moving the container 310 along a rotating trajectory 330 toward the penetrating instrument 320 can act to dispense the content 340 of the container 310 through nozzle 360. Notably, since the container 310 can include more than one internal chamber, more than one chemical can be sprayed through the nozzle 360 onto a target zone of a patient simultaneously.

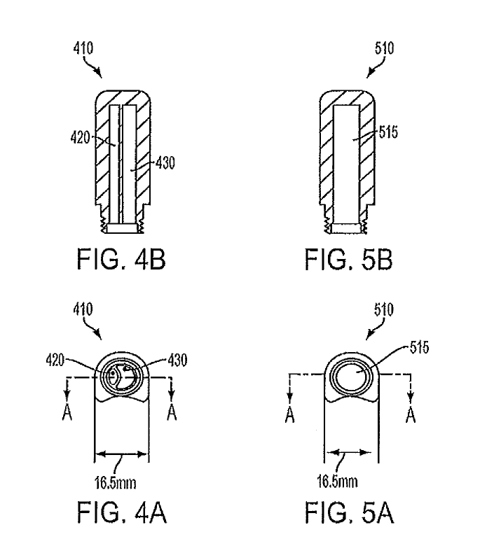

Finally, FIG. 4B provides a cross-sectional view of the interior of an anesthetic and antiseptic dispensing device article of manufacture taken along the A-A line of FIG. 4A. The anesthetic and antiseptic dispensing device article of manufacture can include a container 410. The container 410 can include a first chamber 420 and a second chamber 430 separate from the first chamber 420. Preferably, one of the chambers 420, 430 can contain a pressurized form of a topical anesthetic such as ethyl chloride and the other of the chambers 420 or 430 can contain an antiseptic or other chemical, for instance an analgesic. For example, the container 410 can contain alcohol in one of the chambers 420 or 430 and ethyl chloride in the other of the chambers 420 or 430 for dual topical anesthetic and antiseptic use before introducing a penetrating instrument to the target zone of the patient. It will be recognized by one of skill in the art that the chambers 420, 430 can contain equivalent volumes or can contain different volumes. In another embodiment, FIG. 5B provides a cross-sectional view of the interior container of an anesthetic and antiseptic dispensing device article of manufacture taken along the A-A line of FIG. 5A. The anesthetic and antiseptic dispensing device article of manufacture can include a container 510, which has a single chamber 515 that contains a combination of ethyl chloride and alcohol, or ethyl chloride and povidone-iodine.

I claim:

1. A topical anesthetic and antiseptic dose dispensing device, comprising:
a scalpel having an elongated body with first and second distal ends, at least one of the distal ends comprising a penetrating edge;
a container externally coupled to the elongated body of the scalpel, the container providing at least one chamber, wherein the at least one chamber comprises a single chamber containing a pressurized combination of an aesthetic and an antiseptic; and
a nozzle connected to the container and configured to dispense pressurized content of the at least one chamber responsive to activating a trigger communicating with the nozzle.

2. The device of claim 1, wherein the container provides two chambers, each chamber containing different pressurized content.

3. The device of claim 1, wherein the single chamber contains a pressurized combination of ethyl chloride and one of alcohol and povidone-iodine.

4. The device of claim 2, wherein one chamber contains pressurized ethyl chloride and another chamber contains alcohol.

5. The device of claim 2, wherein one chamber contains pressurized ethyl chloride and another chamber contains povidone-iodine.

6. The device of claim 2, wherein the nozzle comprises a bi-valve nozzle with two openings, each of the two openings corresponding to a different one of the two chambers.

7. The device of claim 1, wherein the container is coupled to the elongated body of the scalpel by way of a trigger.

8. The device of claim 1, wherein the single chamber contains a pressurized combination of ethyl chloride and alcohol.

9. The device of claim 1, wherein the single chamber contains a pressurized combination of ethyl chloride and povidone-iodine.
